(19) United States
(12) Patent Application Publication
(10) Pub. No.: US 2010/0087907 A1
(43) Pub. Date: Apr. 8, 2010

(54) Apparatus And Methods For Treating The Aorta

(75) Inventor: Omar Lattouf, Atlanta, GA (US)

Correspondence Address: FISH & RICHARDSON P.C., P.O BOX 1022, Minneapolis, MN, 55440-1022, US

(73) Assignee: EMORY UNIVERSITY, Atlanta, GA (US)

(21) Appl. No.: 12/527,248

(22) PCT Filed: Feb. 15, 2008
(86) PCT No.: PCT/US08/54133

§ 371 Date: Oct. 29, 2009

Related U.S. Application Data
(60) Provisional application No. 60/890,355, filed on Feb. 16, 2007.

(51) Int. Cl. A61F 2/84; A61F 2/82

(52) U.S. Cl. 623/1.11; 623/1.24; 623/1.15

(57) ABSTRACT

An endovascular stent and an aortic valve replacement can be used to treat the aorta. An endovascular stent suitable for treating the ascending aorta includes a circumferential wall with one or more portions configured to allow blood to flow therethrough, and one or more portions configured to be substantially impermeable. In one version, the blood-permeable portion is defined by bare metal with fenestrations defined therein, and the impermeable portion is formed from polymeric material. The permeable and impermeable portions of the stent are sized, shaped, and located so that, when positioned in the blood vessel to be treated, the impermeable portion is in operative proximity to an aneurysm, dissection, or other area to be treated, and the permeable, bare metal portion is in operative proximity to other blood vessels which need to receive blood from the blood vessel being treated. An aortic valve replacement includes multiple flanges that can be seated in the sinuses of valsalva. The stent or aortic valve replacement may be included in a suitable apparatus for treatment of the aorta. The apparatus for treating the aorta may include a delivery system adapted to be inserted through the apex of the left ventricle of the heart.

### TECHNICAL FIELD

This disclosure relates to surgical apparatus and related methods.

### BACKGROUND

Aortic aneurysms and aortic dissections may occur in the descending aorta (referred to as thoracic or abdominal aneurysms and dissections), in the ascending aorta, or in the transverse arch of the aorta.

One of the features of an aortic dissection is a tear in the intimal layer of the aorta, followed by formation and propagation of a subintimal hematoma (blood clot). The hematoma may occupy a significant percentage, and occasionally all, of the available circumference of the aorta. This also produces a false lumen or double-barreled aorta, which can reduce blood flow to the major arteries arising from the aorta. In the case of the dissection related to the pericardial space, cardiac tamponade other detrimental conditions may result. Aortic aneurysms risk rupture and similar complications.

Aortic aneurysms and dissections currently may be surgically repaired, a procedure which generally requires a surgeon to open the chest cavity, clamp off the aorta, and repair the aneurysm or dissection, such as by sewing a fabric tube, called a graft, to the site. Frequently repair of extensive ascending aortic dissection requires cooling the patient to profound hypothermic level, 18-19 degrees centigrade, in order to allow shutting the blood circulation down; a process called hypothermic circulatory arrest. This enables the surgeon to repair the aortic dissection when it extends into the distal ascending aorta or transverse arch.

It has been proposed to treat thoracic aortic aneurysms by endovascular repair. Such proposals involve insertion of a suitable endoscopic device through the femoral artery, and making a retrograde insertion of an endovascular graft into the descending aorta.

Femoral, retrograde insertion may be contemplated for thoracic aorta aneurysms because of the location of the descending aorta relative to the femoral artery. However, such insertion techniques cannot readily extend into the transverse arch or around the transverse arch and into the ascending aorta.

### SUMMARY

A stent and/or an aortic valve replacement can be used to treat the aorta. The stent includes a circumferential wall with one portion having apertures defined therein. The apertures permit blood to flow through the circumferential wall at that portion. Another portion of the circumferential wall is configured so that blood substantially does not flow through such portion.

In one variation of the stent, the blood-permeable portion is defined by a bare metal frame with fenestrations therein, and the non-permeable portion is defined by a cover over the frame made of suitable material, such as a non-permeable polymeric material.

The size, configuration, and location of the permeable and non-permeable portions of the circumferential wall may be varied, depending upon the particular application. In one implementation, the stent has permeable, bare metal portions at both of its opposite ends and a covered portion located between these ends. The two bare metal portions and the covered portion are in the form of three, adjacent longitudinal segments. Another possible configuration of the stent includes flanges extending from one of the ends of the stent. The flanges are located and configured to engage the sinuses of valsalva.

Still another possible implementation is to have two longitudinal segments defined on the circumferential wall, one having apertures defined therein, and the other being substantially impermeable to blood flow.

An apparatus for treatment of the aorta includes a suitable delivery system for an endovascular stent, the stent being suitably configured for the particular site in the aorta to be treated. One suitable delivery system is adapted for transapical insertion of the stent. The delivery system includes a transapical valve conduit and a delivery device. One possible implementation of the delivery device includes a delivery tube adapted to carry the stent, and a controller for positioning the stent relative to the aorta. Depending on the application and treatments required, the apparatus may include an outer aortic sheath adapted to be positioned on the outer aortic wall, either in conjunction with the stent, or separately therefrom.

The aortic valve replacement can be used to treat the aorta alone, or in combination with a stent. The aortic valve replacement includes a generally cylindrical member comprising a valve located within the generally cylindrical member and multiple flanges at an end of the generally cylindrical member. The flanges can be configured to be positioned into the sinuses of valsalva.

The aortic valve replacement can be inserted by various methods described herein such as by inserting a delivery device carrying an aortic valve replacement through the apex of the left ventricle, advancing a distal end of the delivery device from the left ventricle into the aorta, and controlling the delivery device to position the aortic valve replacement at a desired location on the inner wall of the aorta. The aortic valve replacement can also be provided to the aorta by providing an aortic valve replacement, the aortic valve replacement comprising a valve and multiple flanges at one of the ends, and positioning the flanges of the aortic valve replacement in the sinuses of valsalva of the aortic valve.

An apparatus for providing an aortic valve replacement, comprises an aortic valve replacement formed of a generally cylindrical member and having a valve located within the generally cylindrical member and multiple flanges at an end of the generally cylindrical member, and a delivery system for transapical insertion of the aortic valve replacement, the system comprising a transapical valve conduit and a delivery device, the delivery device having a delivery tube adapted to carry the aortic valve replacement and a controller for positioning the aortic valve replacement relative to the aorta, the controller including a shaft with a first distal end secured to the aortic valve replacement and second end extending toward the proximal end of the delivery tube.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of one implementation of an endovascular stent positioned in the human heart (with a portion of the ascending aorta cut away to show the stent positioned therein);

FIG. 2 is a perspective view of the stent of FIG. 1 showing an exploded view relative to the aorta;

FIG. 3 is a perspective view of an apparatus for treatment of the aorta including a delivery system associated with the stent of FIGS. 1 and 2;

FIG. 4 is a perspective view of the stent of FIGS. 1-2 in relation to a portion of the delivery system of FIG. 3;

FIG. 5 is a perspective view of an outer aortic sheath;

FIG. 6 is a partial, sectional view of the sheath of FIG. 5 deployed with the stent of FIGS. 1-2;

FIG. 7 is a perspective view of an alternative embodiment of a stent;

FIG. 8 is a perspective view of an artificial aortic valve;

FIG. 9 is a perspective view of the artificial aortic valve of FIG. 8 showing an exploded view relative to the aorta; and

FIG. 10 is a perspective view of the stent of FIG. 1 and the artificial aortic valve of FIG. 8 showing an exploded view relative to each other and relative to the aorta.

FIG. 11 is a perspective view of the stent of FIG. 7 that includes a valve and flanges.

Like reference symbols in the various drawings indicate like elements.

### DETAILED DESCRIPTION

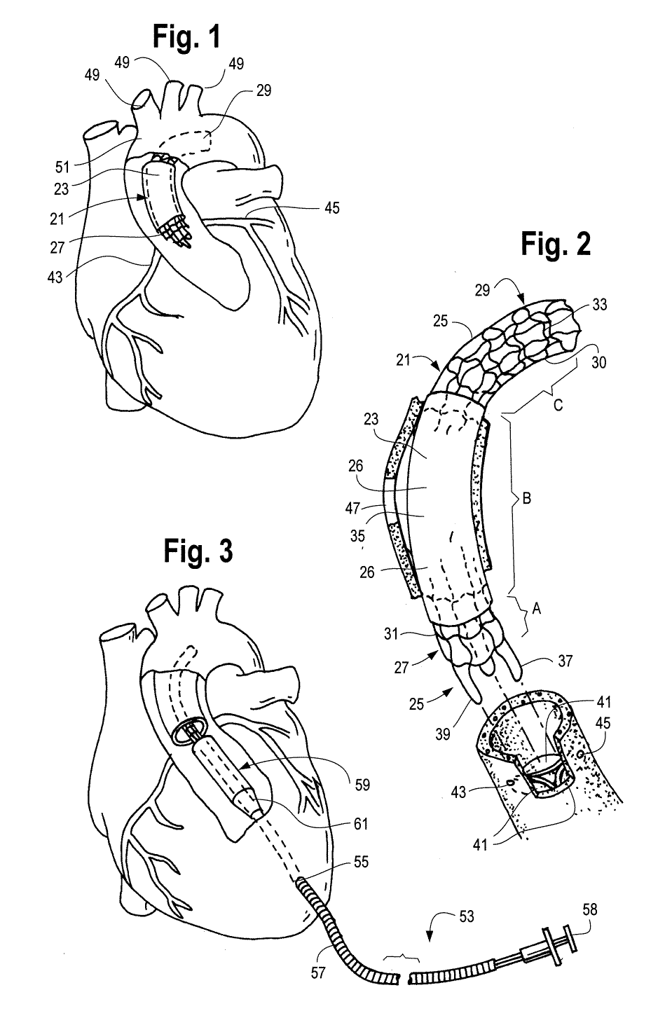

Referring now to the drawings, and in particular to FIGS. 1 and 2, an apparatus for treating the aorta includes a stent 21 having a circumferential wall 23 extending longitudinally between two opposite ends 25, a proximal end 27, and a distal end 29. The stent 21 can be made of a semi-rigid material or a more flexible material that has flexibility properties similar to that of the aorta. Circumferential wall 23 has a portion with apertures defined therein, such portion in this implementation including two, longitudinal, bare metal segments, 31, 33, one at proximal end 27 and the other at distal end 29. The apertures defined in bare metal portions 31, 33 are sized to permit blood to flow therethrough, i.e., they are blood-permeable. The bare metal can be formed of stainless steel or Nytinol.

A second portion 35 of circumferential wall 23 is configured so that blood substantially does not flow through it (blood-impermeable or blood-non-permeable). Portion 35 may be formed of any suitable nonporous material, such as a flexible, polymeric material.

In one possible implementation, circumferential wall 23 is formed by a metal frame 30 having fenestrations defined therein, the frame extending between opposite ends 25 of stent 21. A medial or central portion of the frame carries a cover 26 of polymeric material to define a covered portion 30. The uncovered portions of frame 30, that is, the bare metal portions, include segments 31 and 33. In this implementation, then, proximal bare metal portion 31, intermediate covered portion 35, and distal bare metal portion 33, are adjacent longitudinal segments of lengths A, B, and C, respectively, and located between ends 25 of circumferential wall 23.

Flanges 37 extend from one of the ends 25 of stent 21, in this case, proximal end 27. Although the flanges 37 may assume a variety of forms, in this implementation they are in the form of pods 39 formed from loops of wire. Flanges 37 are located and configured to engage the sinuses of valsalva 41. More particularly, pods 39 extend outwardly from proximal end 27 at spaced locations along the edge of circumferential wall 23, separated by approximately 120° of arc. This configuration permits the outer ends of pods 39 to engage but not obstruct corresponding ones of the three sinuses of valsalva shown in FIG. 2.

When seated in the sinuses of valsalva, stent 21 is configured such that bare metal portion 31 spans but does not obstruct the left main and right coronary arteries 43, 45 (FIG. 1). Covered portion 35, in turn, is located and sized in operative proximity to the portion of the aorta affected by an aneurysm, dissection, or other trauma or distress to the aortic wall. As shown in FIG. 2, operative proximity in this case involves opposing a dissection 47 and having the ends of covered portion 35 extend sufficiently beyond the dissection to span it and reestablish blood flow through the inner wall of the aorta. Other conditions or treatments may involve other placements of stent 21 relative to the area to be treated, such other placements nonetheless being considered in operative proximity if they address or treat the condition of the aortic wall.

Bare metal portion 33 extends outwardly in the distal direction from covered portion 35. In this implementation, bare metal portion 33 is sized, located, or otherwise configured to span at least one of the arteries 49 extending from transverse arch 51. Arteries 49 include the innominate artery, the left common carotid artery, and the left subclavian artery. It will be appreciated that by configuring stent 21 so that bare metal portions 31 and 33 are in operative proximity to arteries extending from the aorta, the apertures defined in such portions permit blood to flow from the aorta into such arteries.

It will likewise be appreciated that the exact lengths, locations, and even configurations of permeable and impermeable portions on circumferential wall 23 may be varied, customized, or otherwise altered to fit any number of endovascular applications, depending on the configuration of the arteries involved, or the location of the aneurysm or dissection relative to such arteries. Similarly, the overall length of stent 21, as well as the overall length of covered portion 35, may be varied or customized depending on the nature and size of the dissection, aneurysm, or other aortic condition to be treated.

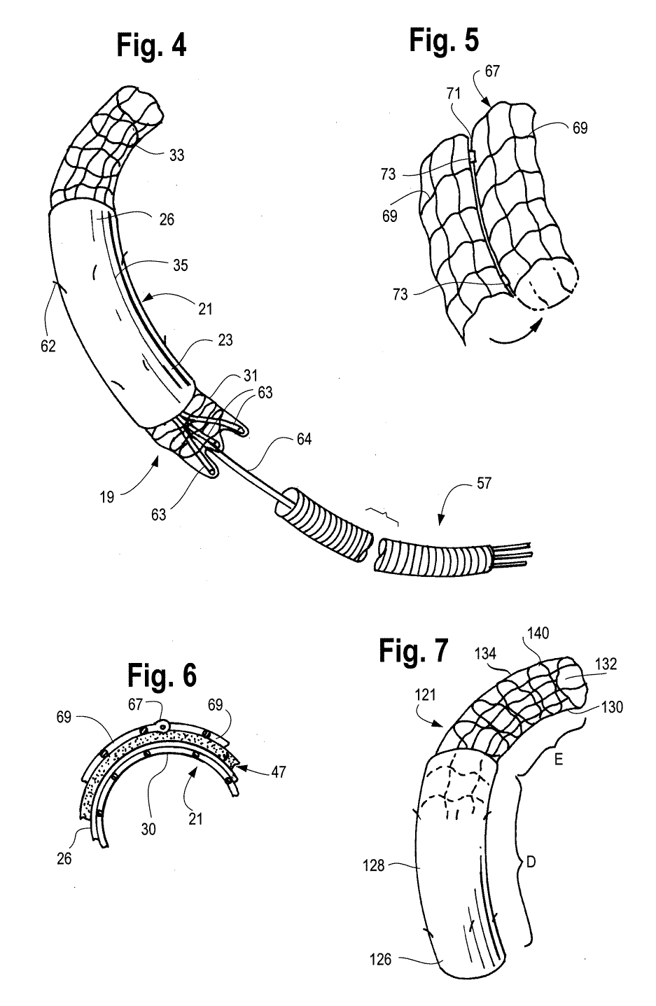

Stent 21 may be delivered to the ascending aorta to be treated by any suitable delivery system through any suitable entry point of the body. Referring now to FIGS. 3 and 4, one suitable delivery system 53 is adapted for insertion of a suitably configured stent through a transapical valve conduit 55 in or near the apex of the left ventricle of the heart. One suitable transapical valve conduit is disclosed in U.S. Pat. No. 6,978,176, the teachings of which are incorporated herein by reference. A delivery device 57 carries the stent in a folded configuration in a delivery tube 61 at the distal end 59 of delivery device 57, and is controlled by a suitable controller 58. Controller 58 may be mechanical, electromechanical, electronic, pneumatic, or any combination thereof suitable for endovascular insertion of stent 21.

As shown in FIG. 4, delivery system 53 includes members 63 with outer ends secured at spaced locations to proximal end 27 of stent 21. Members 63 are connected to shaft 64, which is operatively connected to controller 58. Shaft 64 or members 63 can be suitably operated by controller 58 to control the location and positioning of stent 21 after it has been deployed from delivery tube 61.

For example, pods 39 may be pulled back in the proximal direction by members 63 until they have seated in the sinuses of valsalva associated with the aortic valve, as shown in FIG. 2. By locating members 63 to extend radially from a central shaft 64, the aortic valve (FIG. 2) is able to remain substantially closed, facilitating placement of pods 39 in the sinuses of the valsalva. This, in turn, places bare metal portions 31, 33 in operative proximity to certain arteries so that blood may flow into them from the ascending aorta (or from the transverse arch). Similarly, covered portion 35 is positioned in operative proximity to the aneurysm or dissection to improve blood flow, support the inner aortic wall, or otherwise treat the affected site. Further, wires 62 extending from the covered portion, e.g. by protruding through the covered portion 35, help to prevent distal migration of the stent 21 in the aorta.

Although exemplary, transapical surgical methods have been described with reference to stent 21 and delivery system 53, other delivery systems and even other stents may be used transapically, depending on the particular application. Furthermore, stent 21 and alternative implementations of stent 21 are not limited to being delivered transapically. Stent 21 and variations thereof may be inserted through other entry points, by means of other surgical procedures, into other parts of the aorta, other blood vessels, other anatomical systems, and so on.

For certain courses of treatment, it may be desirable for apparatus 19 to include an optional outer aortic sheath 67, one implementation of which is shown in FIGS. 5 and 6. Sheath 67 includes two semicircular walls 69, movably connected along opposing edges 71 by one or more hinges 73. Sheath 67 is sized, shaped and otherwise configured so that walls 69 can be applied on the outer aortic wall. When used to treat an aneurysm or dissection, the pair of semicircular cylindrical walls 69 can be closed around the site of the aneurysm or dissection and thus function as a support system for the external aortic wall. Depending on the condition, support of the external aortic wall at the site of an aneurysm or dissection may alleviate a distended, weakened, or other medical condition affecting the outer aortic wall. Outer sheath 67 may be applied through open surgery, minimally invasive, or endoscopically aided surgery.

In certain applications, it may be desirable to use outer aortic sheath 67 in conjunction with an endoaortic stent, such as stent 21. For certain conditions, it may be appropriate for stent 21 and sheath 67 to be positioned on opposite sides of the aorta, such as on opposite sides of the site of the aneurysm or dissection. FIG. 6 shows such an application. Frame 30 and outer covering 26 of endoaortic stent 21 opposes the inner wall of the aorta, in operative proximity to a dissection or aneurysm 47. Circumferential walls 69 of sheath 67 engage the outer aortic wall opposite the location of stent 21. In other cases, the condition of the aorta may warrant that stent 21 and sheath 69 be longitudinally spaced or otherwise offset from each other, so as to form either a partial or no overlap between stent 21 and sheath 69.

A number of implementations of the invention have been described. Nevertheless, it will be understood that various modifications may be made without departing from the spirit and scope of the invention. For example, it will be appreciated that the size, shape, and location of the blood-permeable and blood-nonpermeable portions of the stent may be varied to account for any number of factors, including the location of the site to be treated, especially in relation to other arteries leading from the aorta, the condition being treated, the location of the dissection, aneurysm, or other treatment site of the blood vessel walls, and the like. In the implementation shown in FIGS. 1-2, proximal bare metal portion 31 ranges, approximately, from 2 mm to 30 mm in length; covered portion 35 ranges, approximately, from 10 mm to 50 mm in length; and distal bare metal portion 33 ranges, approximately, from 2 mm to 100 mm in length. Again, other lengths and configurations are possible.

Stent 121 shown in FIG. 7 is just one of the many alternate, possible implementations of stents having both blood-permeable and blood-impermeable portions. In this implementation, stent 121 has a metal frame 130, as in stent 21, with fenestrations 132 defined by bare metal portions 134 of the frame 130. Fenestrations 132 are sufficiently sized to permit blood to flow therethrough.

A portion of frame 130 is provided with a cover 126. In this way, stent 121 includes a substantially impermeable longitudinal segment 128 extending over a longitudinal length D, and a blood-permeable segment 140 having length E. In use, stent 121 could be delivered through any suitable endovascular or open surgical procedure and positioned within the blood vessel so that covered, nonpermeable portion 128 is in operative proximity (such as opposing) the area to be treated on the blood vessel wall, whereas bare metal, blood-permeable portion 140 is located in operative proximity to arteries, other blood vessels, valves, ducts, or other openings through which blood or other fluid must flow.

Although the apparatus and associated methods have been discussed with reference to treating the aorta, it will be appreciated that other arteries, blood vessels, and anatomical features may be treated with the stent, the delivery system, or the associated apparatus. It will likewise be appreciated that transapical delivery of the stent is just one way of locating the stent for treatment, and that other insertion techniques and other delivery systems are also suitable.

In some aortic dissections, the dissection can involve the aortic valve and the aortic valve can become incompetent. Furthermore, aortic dilation or failures of the valve itself such as when the annulus loses support can result in the aortic valve becoming incompetent. In these circumstances, the aortic valve can be replaced by an aortic valve replacement.

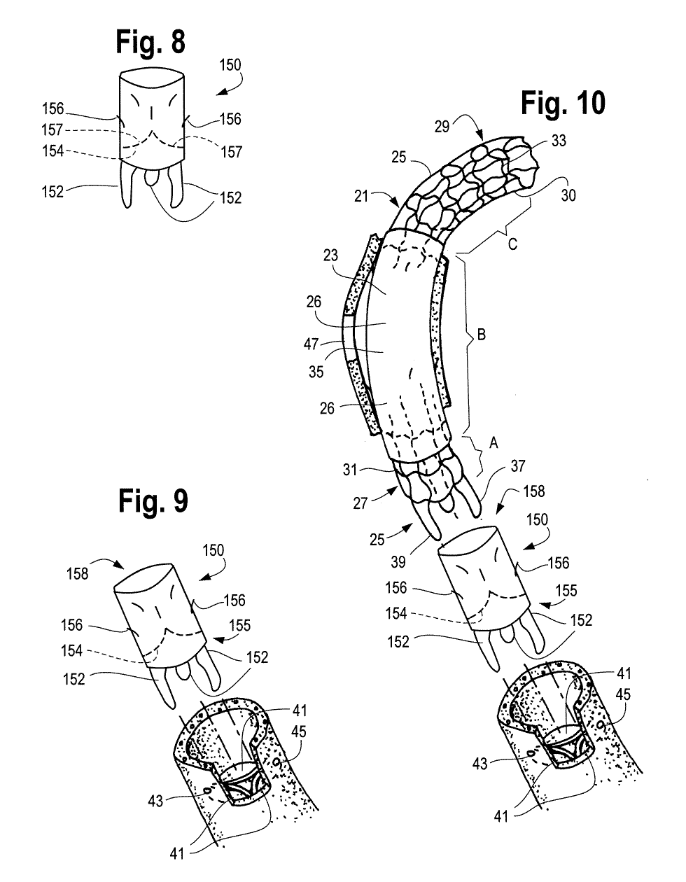

FIG. 8 illustrates an aortic valve replacement 150 that can be used if the aortic valve becomes incompetent. As shown in FIG. 8, the valve replacement 150 includes flanges 152 and a valve 154. The aortic valve replacement 150 is a generally cylindrical member and can be formed of a polymeric material such as Dacron or Gore-Tex® or a metal such as stainless steel or Nytinol. The aortic valve replacement 150 can also include protruding wires 156 that help to prevent distal displacement of the valve replacement in the aorta. The valve 154 is illustrated in FIG. 8 as having two flaps 157 but typically can include three or possibly more flaps. As shown in FIG. 8, the flanges 152 extend from the proximal end 155 of the valve replacement 150. Although the flanges 152 may assume a variety of forms, in this implementation they are in the form of pods formed from loops of wire.

As shown in FIG. 9, the flanges 152 are located and configured to engage the sinuses of valsalva 41. More particularly, the flanges 152 extend outwardly from the proximal end 155 of the valve replacement 150 at spaced locations separated by approximately 120° of arc. This configuration permits the outer ends of the flanges 152 to engage corresponding ones of the three sinuses of valsalva shown in FIG. 9. When seated in the sinuses of valsalva, valve replacement 150 is configured such that the proximal end 155 of the valve replacement spans, but does not obstruct, the left main and right coronary arteries 43, 45 (FIG. 1).

The valve replacement 150 can be inserted into the ascending aorta to be treated by any suitable delivery system through any suitable entry point of the body. For example, the valve replacement 150 can be inserted in the manner illustrated in FIGS. 3 and 4 and described herein with regard to insertion of the stent 21. Alternatively, other delivery systems and even other valve replacements may be used transapically, depending on the particular application. Furthermore, valve replacement 150 and alternative implementations of valve replacement 150 are not limited to being delivered transapically. Valve replacement 150 and variations thereof may be inserted through other entry points, by means of other surgical procedures, into other parts of the aorta, other blood vessels, other anatomical systems, and so on.

As shown in FIG. 10, the aortic valve replacement 150 can be used with the stent 21 described herein. This can occur after the stent 21 has been inserted in the aorta if it is later discovered that the aortic valve is incompetent and needs to be replaced. Alternatively, it may be determined that the stent 21 and aortic valve replacement 150 both need to be inserted within the aorta in the same surgical procedure.

If the aortic valve replacement 150 is inserted after the stent 21 has already been inserted in the aorta, the aortic valve replacement is guided into the aorta using the procedures described herein. The aortic valve replacement 150 can be designed to snugly fit around the proximal end 27 of the stent 21. The aortic valve replacement 150 can cover a significant portion or the proximal portion A of the stent 21 or substantially all of the proximal portion of the stent such that distal end 158 of the aortic valve replacement covers the proximal portion of the stent. The flanges 152 of the aortic valve replacement 150 can be configured to be substantially collinear with the flanges 37 of the stent 21 as the aortic valve replacement is guided over the proximal portion A of the stent. Once the aortic valve replacement 150 and the stent 21 are engaged, the flanges 152 can be positioned to engage and seat within the sinuses of valsalva as discussed herein. For example, the aortic valve replacement 150 and the stent 21 can be pulled back together toward the aortic valve to position the flanges 152 within the sinuses of valsalva.

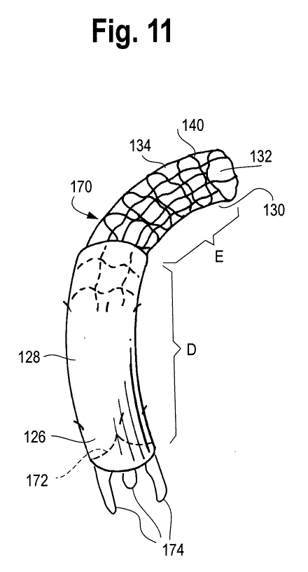

As shown in FIG. 11, the stent 21 and aortic valve replacement 150 can be provided as a unitary stent 170 by including a valve 172 and flanges 174 in a stent structure like the one illustrated in FIG. 7. The stent 170 can be installed in the manner described herein for the stent 21 and aortic valve replacement 150.

The outer sheath 67 described herein and illustrated in FIGS. 5 and 6 can also be used in some situations with the valve replacement 150, where outer support of the aorta is needed. For example, the outer sheath 67 can be used with the configuration illustrated in FIG. 9 or the configuration illustrated in FIG. 10 to help support the aorta. Circumferential walls 69 of sheath 67 can engage the outer aortic wall opposite the location of aortic valve replacement 150. In other cases, the condition of the aorta may warrant that the aortic valve replacement 150 and the sheath 69 be longitudinally spaced or otherwise offset from each other, so as to form either a partial or no overlap between the aortic valve replacement 150 and the sheath 69.

Accordingly, other implementations of the apparatus, stents, valve replacements and the delivery systems disclosed herein are within the scope of the following claims.

What is claimed is:

1. A method of treating the aorta comprising:
inserting a delivery device carrying a stent through the apex of the left ventricle;
advancing a distal end of the delivery device from the left ventricle into the aorta; and
controlling the delivery device to position the stent at a desired location on the inner wall of the aorta.

2. The method of claim 1, wherein inserting the delivery device includes inserting the device through a one-way, transapical valve conduit.

3. The method of claim 1, wherein controlling the delivery device includes positioning the stent in one of the ascending aorta and the transverse arch of the aorta.

4. The method of claim 1, wherein the stent is positioned at a location associated with at least one of an aneurysm and a dissection.

5. The method of claim 1, including providing a stent with apertures defined in a circumferential wall of the stent, the apertures sized to permit blood flow therethrough, and positioning the stent within the aorta to locate the apertures in proximity to other arteries receiving blood from the aorta.

6. The method of claim 5, further including:
providing the stent with a longitudinal segment on the circumferential wall without apertures defined therein;
positioning the longitudinal segment within a portion of the aortic wall to be treated; and
positioning the apertures adjacent at least one artery receiving blood from the aorta.

7. The method of claim 1, further including:
providing members for positioning the stent; and
securing the members at spaced locations on the stent;
wherein controlling the delivery device includes operating the members to position the stent at a desired location.

8. The method of claim 1, further comprising:
positioning an aortic valve replacement between the stent and the sinuses of valsalva.

9. The method of claim 8, wherein said positioning step comprises:
inserting a delivery device carrying an aortic valve replacement through the apex of the left ventricle;
advancing a distal end of the delivery device from the left ventricle into the aorta at a location between the stent and the sinuses of valsalva; and
controlling the delivery device to position the aortic valve replacement between the stent and the sinuses of valsalva.

10. The method of claim 8, wherein the aortic valve replacement includes multiple flanges and the flanges are positioned within the sinuses of valsalva.

11. The method of claim 10, wherein the stent includes multiple flanges and the flanges of the aortic valve replacement are directed to be substantially collinear with the flanges of the stent.

12. A method of treating an aortic condition, the method comprising:
providing a stent, the stent having a circumferential wall with at least two zones defined therein, the first zone of the circumferential wall having apertures defined therein, the apertures sized to permit blood to flow therethrough, the second zone of the circumferential wall through which blood substantially does not flow; and
positioning the stent near the aortic valve to locate the first zone in operative proximity to the left main and right coronary arteries, and the second zone in operative proximity to the aortic condition.

13. The method of claim 12, wherein the aortic condition is an aortic dissection or aneurysm.

14. The method of claim 12, wherein the stent has two ends, the circumferential wall extending between the two ends, and wherein the stent is provided with multiple flanges at one of the ends, the method further including seating the stent by positioning the flanges in the sinuses of valsalva of the aortic valve.

15. The method of claim 12, further including carrying the stent through the left ventricle and into the ascending aorta on a distal end of a controllable delivery device.

16. The method of claim 15, further including:
folding the stent into a delivery tube at the distal end of the delivery device; and
securing members at spaced locations on the stent.

17. The method of claim 16, wherein positioning the stent includes moving the delivery tube relative to the dissection.

18. The method of claim 16, wherein positioning the stent includes moving the members.

19. The method of claim 18, wherein positioning the stent includes seating the stent by pulling the members back toward the aortic valve.

20. The method of claim 12, further including applying a cylindrical sheath to the outer aortic wall of the ascending aorta at the site of the dissection.

21. A stent, comprising:
a circumferential wall extending longitudinally between two opposite ends;
a first portion of the circumferential wall having apertures defined therein, the apertures sized to permit blood to flow therethrough; and
a second portion of the circumferential wall configured so that blood substantially does not flow therethrough.

22. The stent of claim 21, wherein the first portion comprises bare metal.

23. The stent of claim 22, wherein the apertures include fenestrations defined by portions of the bare metal.

24. The stent of claim 21, wherein the second portion includes flexible, polymeric material impermeable to fluid.

25. The stent of claim 21, wherein the circumferential wall comprises a frame extending at least partially between the ends, the frame having fenestrations defined therein, the frame partially covered by a polymeric material to define a covered portion and a bare metal portion, the first portion of the circumferential wall corresponding to the bare metal portion, the second portion corresponding to the covered portion.

26. The stent of claim 21, wherein the first and second portions comprise first and second longitudinal segments of the circumferential wall.

27. The stent of claim 26, wherein the first and second longitudinal segments are adjacent to each other.

28. The stent of claim 27, wherein the first segment is located at one of the ends of the circumferential wall.

29. The stent of claim 28, wherein the first segment extends a sufficient length to span the left main and right coronary arteries when the stent is seated in the sinuses of valsalva.

30. The stent of claim 23, wherein the first portion includes two, longitudinal, bare metal segments, one at a proximal end portion of the stent, the other at a distal end portion of the stent.

31. The stent of claim 30, wherein one of the bare metal segments is of sufficient length to span at least one of the arteries extending from the transverse arch.

32. The stent of claim 30, wherein the second portion includes a covered, longitudinal segment between the two bare metal segments, the three segments being sized and located so that, when the stent is located adjacent the aortic valve, the first bare metal segment spans the coronary arteries and the second bare metal segment at least partially spans the three arteries extending from the transverse arch comprising the innominate artery, the left common carotid, and the left subclavian.

33. The stent of claim 21, further comprising flanges extending from one of the ends of the stent, the flanges located and configured to engage the sinuses of valsalva.

34. The stent of claim 33, wherein the flanges comprise pods formed from loops of wire.

35. The stent of claim 21, further comprising an outer aortic sheath adapted to be positioned on the other side of the aortic wall opposite the circumferential wall of the stent.

36. The stent of claim 21, wherein:
the second portion includes a valve and flanges such that, when the stent is located adjacent the aortic valve, that flanges engage the sinuses of valsalva; and
the first portion is distal to the second portion within the aorta.

37. An apparatus for treatment of the aorta comprising:
a stent having an endoaortic circumferential wall extending longitudinally between two, opposite ends, a first portion of the circumferential wall having apertures defined therein, the apertures sized to permit blood to flow therethrough, a second portion of the circumferential wall configured so that blood substantially does not flow therethrough;
a delivery system for transapical insertion of the stent, the system comprising a transapical valve conduit and a delivery device, the delivery device having a delivery tube adapted to carry the stent and a controller for positioning the stent relative to the aorta, the controller including a shaft with a first distal end secured to the stent and second end extending toward the proximal end of the delivery tube; and
an outer aortic sheath adapted to be positioned on the outer aortic wall.

38. A method of treating the aorta comprising:
inserting a delivery device carrying an aortic valve replacement through the apex of the left ventricle;
advancing a distal end of the delivery device from the left ventricle into the aorta; and
controlling the delivery device to position the aortic valve replacement at a desired location on the inner wall of the aorta.

39. The method of claim 38, wherein inserting the delivery device includes inserting the device through a one-way, transapical valve conduit.

40. The method of claim 38, further including:
providing members for positioning the aortic valve replacement; and
securing the members at spaced locations on the aortic valve replacement;
wherein controlling the delivery device includes operating the members to position the aortic valve replacement at a desired location.

41. A method of providing an aortic valve replacement to the aorta, the method comprising:
providing an aortic valve replacement, the aortic valve replacement comprising a valve and multiple flanges at one of the ends, and
positioning the flanges of the aortic valve replacement in the sinuses of valsalva of the aortic valve.

42. The method of claim 41, further including carrying the aortic valve replacement through the left ventricle and into the ascending aorta on a distal end of a controllable delivery device.

43. The method of claim 41, wherein positioning the aortic valve replacement includes securing members at spaced locations on the proximal end of the aortic valve replacement and seating the aortic valve replacement by pulling the members back toward the sinuses of valsalva.

44. An aortic valve replacement, comprising:
a generally cylindrical member comprising a valve located within the generally cylindrical member and multiple flanges at an end of the generally cylindrical member;
wherein the flanges are configured to be positioned into the sinuses of valsalva.

45. The aortic valve replacement of claim 44, including three flanges separated by approximately 120° of arc.

46. An apparatus for providing an aortic valve replacement, comprising:
an aortic valve replacement formed of a generally cylindrical member and having a valve located within the generally cylindrical member and multiple flanges at an end of the generally cylindrical member;
a delivery system for transapical insertion of the aortic valve replacement, the system comprising a transapical valve conduit and a delivery device, the delivery device having a delivery tube adapted to carry the aortic valve replacement and a controller for positioning the aortic valve replacement relative to the aorta, the controller including a shaft with a first distal end secured to the aortic valve replacement and second end extending toward the proximal end of the delivery tube.
